(19) United States
(12) Patent Application Publication
(10) Pub. No.: US 2025/0110548 A1
(43) Pub. Date: Apr. 3, 2025

(54) EXPRESSION ESTIMATION FOR HEADSETS USING LOW-PROFILE ANTENNA AND IMPEDANCE CHARACTERISTIC SENSING

(71) Applicants: Apple Inc., Cupertino, CA (US); Carnegie Mellon University, Pittsburgh, PA (US)

(72) Inventors: Daehwa Kim, Yeongcheon-si (KR); Istvan J. Szini, Santa Clara, CA (US); Christopher Harrison, Pittsburgh, PA (US); Brian H. Tsang, Belmont, CA (US)

(21) Appl. No.: 18/895,924

(22) Filed: Sep. 25, 2024

Related U.S. Application Data
(60) Provisional application No. 63/541,601, filed on Sep. 29, 2023.

(51) Int. Cl. G06F 3/01; G01S 13/89; H01Q 1/27

(52) U.S. Cl. CPC G06F 3/012; G01S 13/89; H01Q 1/276

(57) ABSTRACT

Various implementations disclosed herein include devices, systems, and methods that predict a face configuration or hand pose of a user. For example, a device may include a structure and one or more antennas positioned on a portion of the structure. The device is worn on a head of a user. An associated process may be enabled to interpret data from the one or more antennas to discriminate deformation of a tissue geometry of at least one body portion of a user when the device is worn on the head of the user. The data from the one or more antennas may be associated with one or more impedance characteristics.

### CROSS-REFERENCE TO RELATED APPLICATIONS

This application claims the benefit of U.S. Provisional Application Ser. No. 63/541,601 filed Sep. 29, 2023, which is incorporated herein in its entirety

### TECHNICAL FIELD

The present disclosure generally relates to systems, methods, and devices that determine facial and/or hand pose configurations of users of electronic devices, for example, identifying the three-dimensional (3D) locations of points on a head-mounted device (HMD) user's cheeks, jaw, lips, tongue, a hand(s) in front of a mouth, etc.

### BACKGROUND

Existing techniques for tracking or otherwise determining an electronic device user's facial expressions, facial features, hand pose expressions, etc. may be improved with respect to accuracy, efficiency, and other attributes.

### SUMMARY

Various implementations disclosed herein include devices, systems, and methods that discriminate a user's facial deformations, hand pose configurations (e.g., in front of a mouth), and/or the 3D positions of points on the surface or tissue geometry of the user's head/face and/or hand(s). The facial and/or hand pose information may be used to provide content, such as virtual content, within an extended reality (XR) environment, e.g., a VR or AR environment. A user's facial deformations, hand pose configurations, and/or the 3D positions of points on the surface or tissue geometry of the user's head/face, and/or hand(s) may be discriminated via an XR headset device that utilizes a radio frequency (RF) signal obtained via one or more antennas. Such information may be used to predict a user's facial configuration such as inter alia, 3-dimensional (3D) key-points such as, inter alia, cheeks, jaw, lips, tongue, etc. of the user. Likewise, such information may be used to predict and/or discriminate a user's hand pose configurations and associated expressions.

A headset device may include a head mounted display (HMD), heads up display glasses, AR glasses comprising transparent eye pieces, vision corrective glasses, etc. The one or more antennas may be located on or integrated within a portion of a headset such as, inter alia, a bottom portion of a XR headset device. In some implementations, a user's head/facial/hand features such as, inter alia, a user's facial expression and/or hand pose may dielectrically and contactlessly interact with at least one of the one or more antennas such that changes to a facial configuration and/or hand pose configuration of the user may manifest within a self-resonance frequency and/or performance associated with at least one of the one or more antennas. In some implementations, values of the self-resonance frequency and/or performance may be measured via the XR headset device. Resulting data associated with the user's head/facial/hand pose features may be utilized, for example, to configure a persona, interpret user facial/hand gestures/expressions, and/or to uniquely identify the user. A persona may comprise, inter alia, a photo-realistic representation of the user, an abstracted representation of the user (e.g., an animated representation of the user), any type of avatar, etc.

In some implementations, the one or more antennas are configured to minimize interference generated via user hands, face coverings (e.g., a beard, a facemask, etc.) and/or an associated environment by utilizing a directional radiation pattern associated with an angular dependence of a strength of RF waves emitted from the one or more antennas. Some implementations, utilize, inter alia, a slot antenna, which may be beneficial in some contexts due to its low-profile and simplified structural elements. Some implementations utilize a slot antenna configured to be folded in half, which may provide a location for placement of a secondary antenna. Some implementations utilize a vertically-polarized U-shaped slot antenna placed within a center of an XR headset and a horizontally-polarized antenna placed adjacent to side portion of the XR headset (e.g., a specified distance from an edge of the XR headset). Some implementations include multiple antennas utilizing scattering parameters (any type of S-parameters) comprising vector signals/entities describing electrical behavior of linear electrical networks when undergoing various steady state stimuli by electrical signals. For example, with respect to a 2-port antenna configuration there may be four or more S-parameters (e.g., S11, S21, S12, and S22) and each port may be configured to inject a signal and measure a reflected signal with respect to its own port such as, inter alia, an S11 parameter (input port voltage reflection coefficient indicating power reflected back to a transmitting antenna) or an S22 parameter (output port voltage reflection coefficient) and a transmitted signal from additional ports such as an S21 parameter (forward voltage gain indicating transmitted power between antennas) or an S12 parameter (reverse voltage gain). Specifically, the electrical networks (e.g., the surface of a face or hand) are deformed (e.g., a change in face geometries, different facial expressions, different hand pose expressions, etc.) and an electromagnetic impedance profile of the face and/or hands have additionally been changed. Subsequently, while the incident signal (e.g., from port 1) pass through the networks (e.g., face, hand, etc.), a number of signals being reflected (e.g., back to port 1) and transmitted (e.g., going to port 2) varies and may be measured as S-parameters, inter alia, S11 for profiling a reflected signal and S21 for profiling a transmitted signal. In some implementations, one or more antennas/antenna array may be implemented with controllable-beam steering, therefore modifying the input signal phase with respect to radiating elements controlling the antenna system radiation pattern directivity. Some implementations utilize multiport antenna configurations, such that a matrix of test points may expand based on a number of antennas used. For example, 2 antennas may produce S11, S22, S21 and S12 parameters, 4 or more antennas may produce S11, S22, S33, S44, S12, S13, S14 . . . . Snm, etc. for a total of 16 test points for magnitude and an additional 16 test points for phase.

In some implementations, inputs to a machine learning (ML) model and/or a rule-based model may include, inter alia, S11, S22 . . . . Sn port return loss magnitudes, S11, S22 . . . . Sn port phase shifts, S21 or any other ports combinations magnitudes, S21 or any other ports combinations phase shifts, etc. Some implementations provide additional featurization to be utilized in addition to raw measurements such as a first derivative of a series, a difference from previous frame data, a standard deviation, coefficients from a polynomial fit, subtraction of available ports vectors, etc. Some implementations provide an output comprising a series of 3D key-points, a facial expression classification, and/or a user identification. An interpretation (rule-based) algorithm or ML model and/or additional mechanisms (e.g., using motion sensor input) may be configured to account for head/hand orientations (e.g., titled far forward) in which a user's body (e.g., chest) modifies an associated interference.

In some implementations, a device has a structure, one or more antennas positioned on a portion of the structure, and a processor. The one or more antennas comprises at least one or combined antenna topologies. The processor is configured to interpret data from the one or more antennas to discriminate deformation of a tissue geometry of at least a portion of a user when the device is worn on the head of the user. The data from the one or more antennas may include data associated with one or more impedance characteristics.

In accordance with some implementations, a device includes one or more processors, a non-transitory memory, and one or more programs; the one or more programs are stored in the non-transitory memory and configured to be executed by the one or more processors and the one or more programs include instructions for performing or causing performance of any of the methods described herein. In accordance with some implementations, a non-transitory computer readable storage medium has stored therein instructions, which, when executed by one or more processors of a device, cause the device to perform or cause performance of any of the methods described herein. In accordance with some implementations, a device includes: one or more processors, a non-transitory memory, and means for performing or causing performance of any of the methods described herein.

### BRIEF DESCRIPTION OF THE DRAWINGS

So that the present disclosure can be understood by those of ordinary skill in the art, a more detailed description may be had by reference to aspects of some illustrative implementations, some of which are shown in the accompanying drawings.

FIG. 1 illustrates an exemplary electronic device operating in a physical environment in motion in accordance with some implementations.

FIG. 2 illustrates a system enabled to analyze user facial gestures and/or expressions configure a persona and/or to uniquely identify a user, in accordance with some implementations.

FIG. 3A illustrates an example of a headset device configured to utilize an RF signal obtained via an antenna(s) to predict a user's facial configuration, in accordance with some implementations.

FIG. 3B illustrates an alternative example of a headset device configured to utilize an RF signal obtained via an antenna(s) to predict a user's facial configuration, in accordance with some implementations.

FIG. 4A illustrates an exemplary view in which a user is wearing a headset device enabled to analyze user facial gestures and/or expressions, in accordance with some implementations.

FIG. 4B illustrates an exemplary view of a user being presented facial expressions via a headset device for generating corresponding output personas and associated key points, in accordance with some implementations.

FIG. 4C illustrates an exemplary view in which a user is wearing a headset device enabled to analyze user hand gestures and/or expressions, in accordance with some implementations.

FIG. 4D illustrates an alternative exemplary view in which a user is wearing a headset device enabled to analyze user hand gestures and/or expressions, in accordance with some implementations.

FIG. 5 is a flowchart representation of an exemplary method that utilizes an RF signal obtained via an antenna(s) to predict a user's body portion configuration, in accordance with some implementations.

FIG. 6 is a block diagram of an electronic device of in accordance with some implementations.

In accordance with common practice the various features illustrated in the drawings may not be drawn to scale. Accordingly, the dimensions of the various features may be arbitrarily expanded or reduced for clarity. In addition, some of the drawings may not depict all of the components of a given system, method or device. Finally, like reference numerals may be used to denote like features throughout the specification and figures.

### DESCRIPTION

Numerous details are described in order to provide a thorough understanding of the example implementations shown in the drawings. However, the drawings merely show some example aspects of the present disclosure and are therefore not to be considered limiting. Those of ordinary skill in the art will appreciate that other effective aspects and/or variants do not include all of the specific details described herein. Moreover, well-known systems, methods, components, devices and circuits have not been described in exhaustive detail so as not to obscure more pertinent aspects of the example implementations described herein.

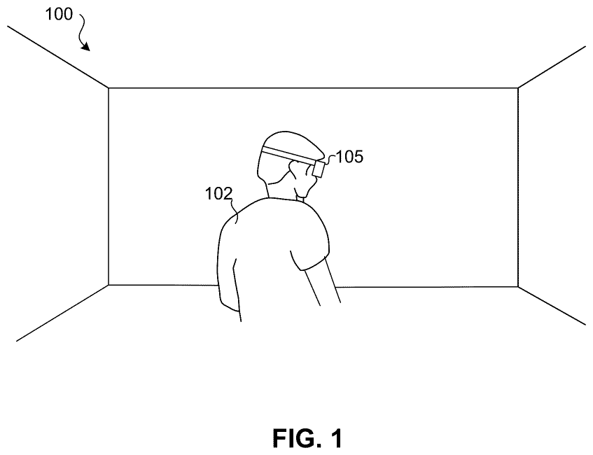

FIG. 1 illustrates an exemplary electronic device 105 operating in a physical environment 100. In the example of FIG. 1, the physical environment 100 is a room. The electronic device 105 may include one or more antennas, one or more cameras, microphones, depth sensors, or other sensors that can be used to capture information about and evaluate the physical environment 100 and the objects within it, as well as information such as facial features and/or hand pose configurations (e.g., in front of a mouth) of the user 102 of electronic devices 105. The information about the physical environment 100 and/or facial features and/or hand pose configurations of the user 102 may be used to provide visual and audio content and/or to identify the current location of the physical environment 100 and/or the location of the user within the physical environment 100. Likewise, the information about the facial features and/or hand pose configurations of the user may be utilized to configure a persona, interpret user facial gestures/expressions, interpret user hand pose gestures/expressions, uniquely identify the user, etc.

In some implementations, views of an extended reality (XR) environment may be provided to one or more participants (e.g., user 102 and/or other participants not shown) via electronic device 105 (e.g., a wearable device such as an HMD, an XR headset, etc.). Such an XR environment may include views of a 3D environment that is generated based on camera images and/or depth camera images of the physical environment 100 as well as a representation of user 102 based on camera images and/or depth camera images of the user 102. Such an XR environment may include virtual content that is positioned at 3D locations relative to a 3D coordinate system (i.e., a 3D space) associated with the XR environment, which may correspond to a 3D coordinate system of the physical environment 100.

In some implementations, video (e.g., pass-through video depicting a physical environment) is received from an image sensor of a device (e.g., device 105). In some implementations, a 3D representation of a virtual environment is aligned with a 3D coordinate system of the physical environment. A sizing of the 3D representation of the virtual environment may be generated based on, inter alia, a scale of the physical environment or a positioning of an open space, floor, wall, etc. such that the 3D representation is configured to align with corresponding features of the physical environment. In some implementations, a viewpoint within the 3D coordinate system may be determined based on a position of the electronic device within the physical environment. The viewpoint may be determined based on, inter alia, image data, depth sensor data, motion sensor data, etc., which may be retrieved via a virtual inertial odometry system (VIO), a simultaneous localization and mapping (SLAM) system, etc.

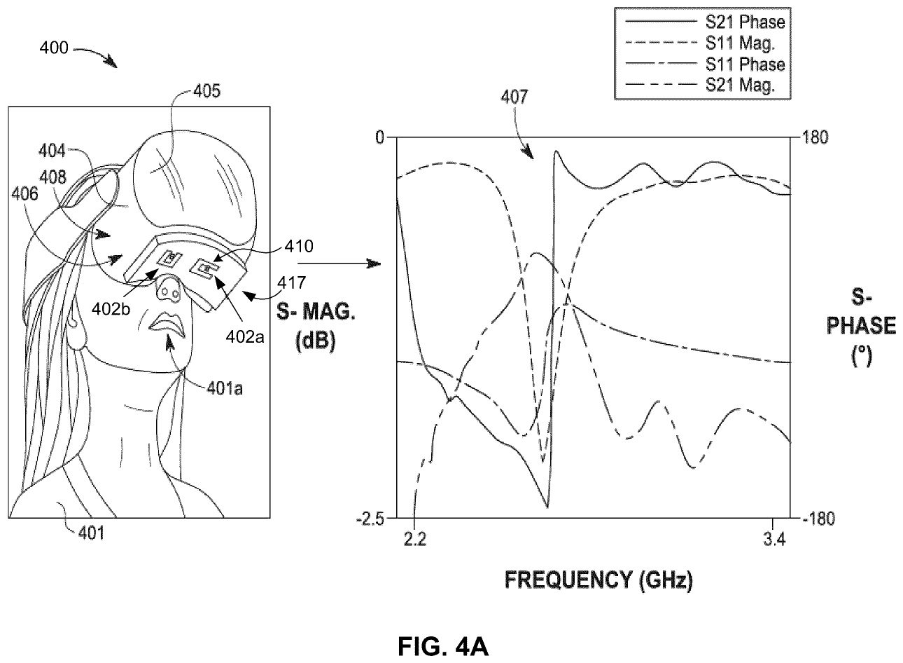

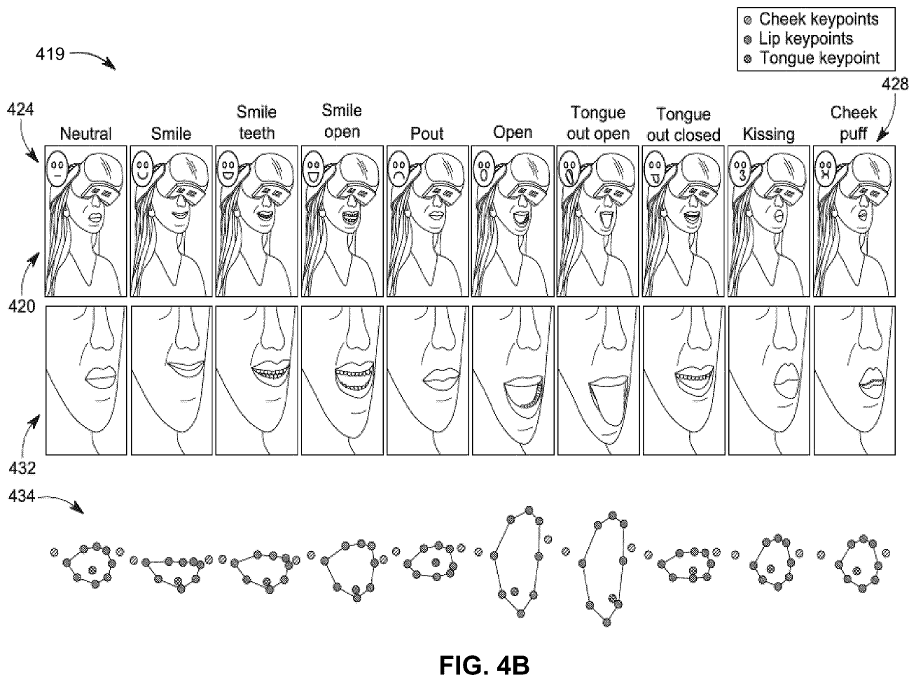

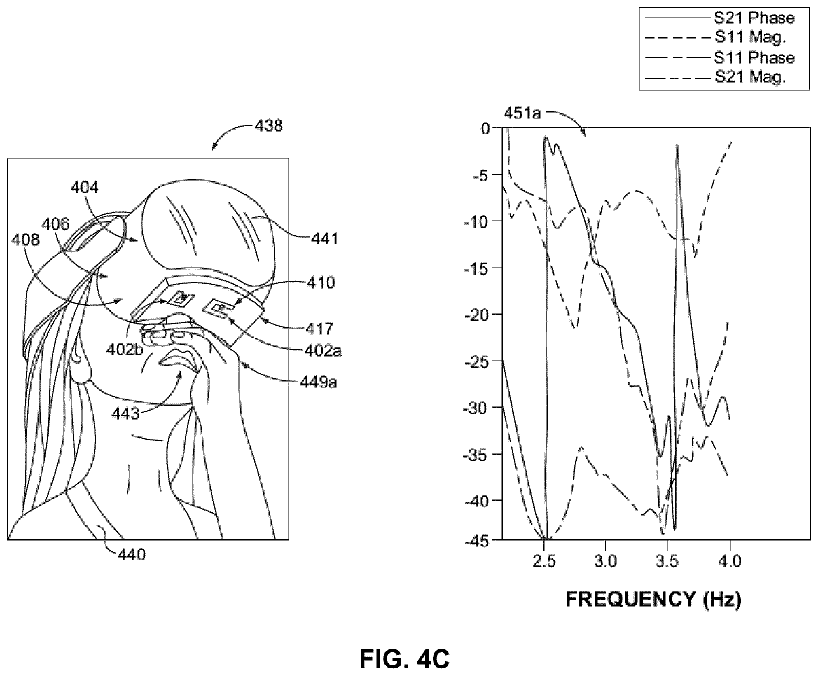

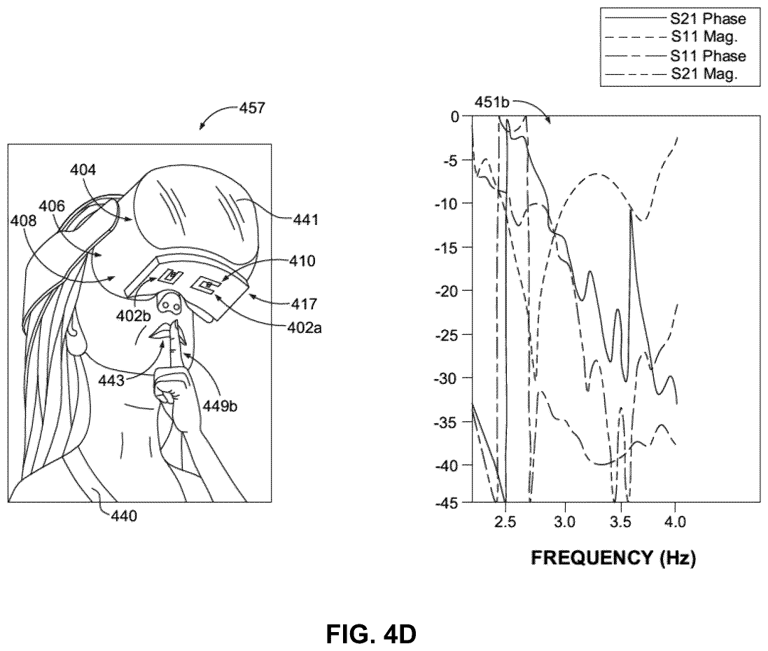

In some implementations, an HMD or heads up display glasses (e.g., device 105), communicatively coupled to a server, or other external device may be configured to capture a user's holistic configuration for utilization within XR environments such as providing multimodal input to generate expressive personas. Capturing a user's holistic configuration may include capturing facial features associated with a user's mouth, such as a mouth configuration and/or capturing hand movement/position configurations associated with a user's hand, to provide a channel for representing face pose information and/or a 3D hand pose expression/interpretation. For example, emotive cues may be obtained via facial expressions such that when nonverbal signals such as facial expressions match situational context and spoken content, trust, clarity, and rapport among users may be improved. In some implementations, an open and closed mouth pose may be potentially used for dietary tracking. Likewise, hand configurations/expressions may be used to obtain emotive cues associated with a specified gesture (e.g., in front of a mouth of a user) such as a silent/quiet gesture as illustrated with respect to FIG. 4D infra.

In some implementations, an HMD or heads up display glasses may enable an RF-based approach for capturing mouth configuration information of a user by utilizing an antenna associated with or integrated into a portion (e.g., an underside portion) of an HMD or heads up display glasses. The aforementioned RF-based approach for capturing mouth/hand configuration information may eliminate privacy issues associated with camera-based facial/hand feature capturing methods while simultaneously supporting silent facial/hand expression determination. Likewise, an RF-based approach for capturing mouth and/or hand configuration information requires no device contact with a user's body and may be fully self-contained within an HMD or heads up display glasses thereby offering a high degree of physical robustness and user practicality.

In some implementations, an HMD (or heads-up display glasses) is configured to sense antenna impedance characteristics. For example, dielectrics, such as human tissue, located within a close distance with respect to an antenna(s) may be loaded as a component of the antenna(s) such that an associated reference ground plane may be affected thereby affecting the antenna(s) characteristic impedance and radiation performance with respect to a given reference frequency. Associated geometry changes within a dielectric (e.g., occurring during movement of a user's mouth or hand) may reflect changes on the perceived ground plane of the antenna(s) and a subsequently varied antenna performance may be measured with respect to S-parameters. The antenna(s) may comprise a low-profile antenna topology such as a sub-millimeter low-profile cross-polarized antenna system providing a suitable structure for integration into an HMD. Likewise, S21 . . . . Snm (multiple antennas) data such as S21 port magnitudes, S21 port phase shifts, etc. may be utilized to quantify a performance gain of the antenna(s). In some implementations, an HMD comprises specialized sensing hardware configured to unlock increased framerates and resolution.

Some implementations utilize electromagnetic simulation computational re-sources to determine antenna system topology designs. Likewise, determining an S-parameter response for sample mouth configurations may include building representative mouth configurations on a 3D human phantom head or hand. For example, representative mouth configurations may include, inter alia, mouth closed, mouth open, smile closed, smile with teeth, open mouth with tongue out, etc. Likewise, determining an S-parameter response for sample hand configurations may include building representative hand configurations on a 3D human phantom hand. For example, a sample hand configuration may include, inter alia, a silent/quiet gesture represented by a finger in front of a mouth of the user. In some implementations, lossy copper material properties may be utilized for antennas comprising different design parameters. Minimizing a mismatch loss between antennas may include implementing an impedance-matching network for each antenna at a predefined frequency of interest.

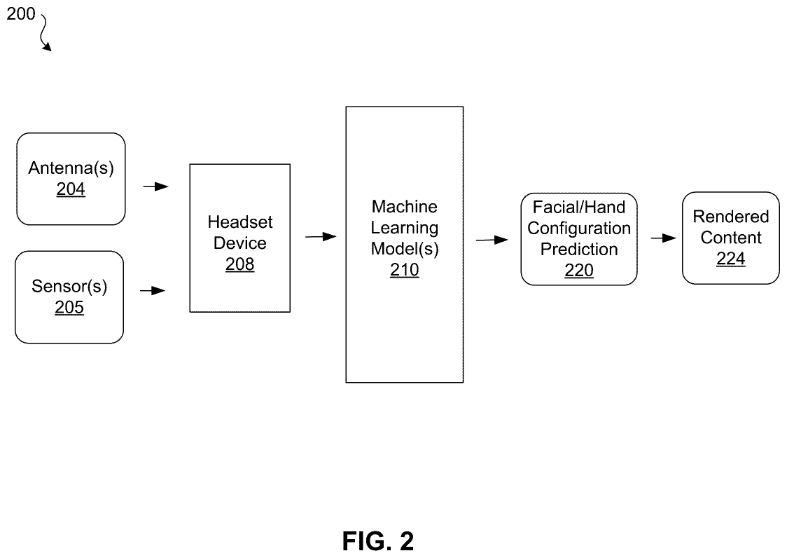

FIG. 2 illustrates a system 200 enabled to analyze user facial and/or hand pose gestures and/or expressions to configure a persona and/or to uniquely identify a user, in accordance with some implementations. System 200 comprises a headset device 208 (e.g., a XR headset device such as an HMD, heads up display glasses, AR glasses comprising transparent eye pieces, vision corrective glasses, etc.) and a machine learning model(s) 210 (or pipeline) configured to analyze antenna data to track user facial and/or hand features for predicting a facial and/or hand configuration such as, inter alia, 3D key points with respect to cheeks, lips, hands, and/or tongue of a user.

System 200 enables a user's facial/hand pose configuration prediction 220 to be generated via headset device 208 utilizing an RF signal obtained via antenna(s) 204. Optional sensors 205 such as inward or outward facing cameras may be used to provide additional data for generating facial/hand pose configuration prediction 220 and/or configuring a subsequent persona. The antenna(s) 204 may be located adjacent to, on, and/or integrated with a bottom portion of headset device 208. Likewise, the antenna(s) 204 may be located adjacent to, on, and/or integrated with any portion of headset device 208. In some implementations, user facial and/or hand features such as, inter alia, a mouth of the user may change the perceived ground plane of the antenna(s) 204 such that changes to a facial configuration of the user may manifest within a self-resonance frequency and/or performance associated with at least one of the one or more antennas. Values of the self-resonance frequency and/or performance may be measured via headset device 208 and a resulting facial/hand pose configuration prediction 220 (e.g., generated via machine learning model 210) associated with the user's facial and/or hand pose features may be utilized, for example, to generate rendered content 224 such as, inter alia, a persona, user facial/hand gestures/expressions, to identify the user, etc.

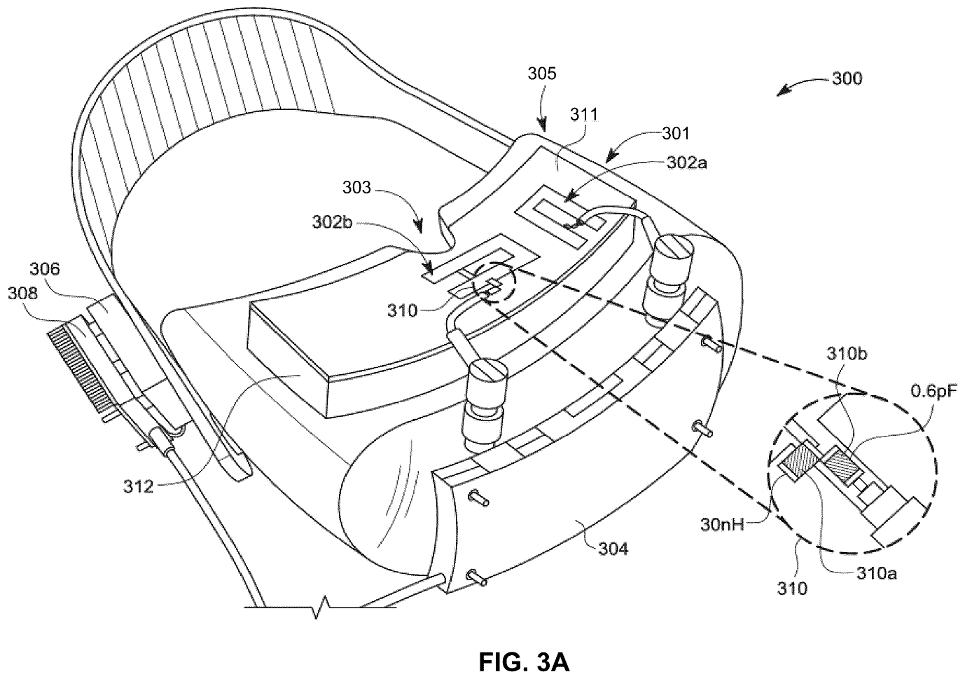

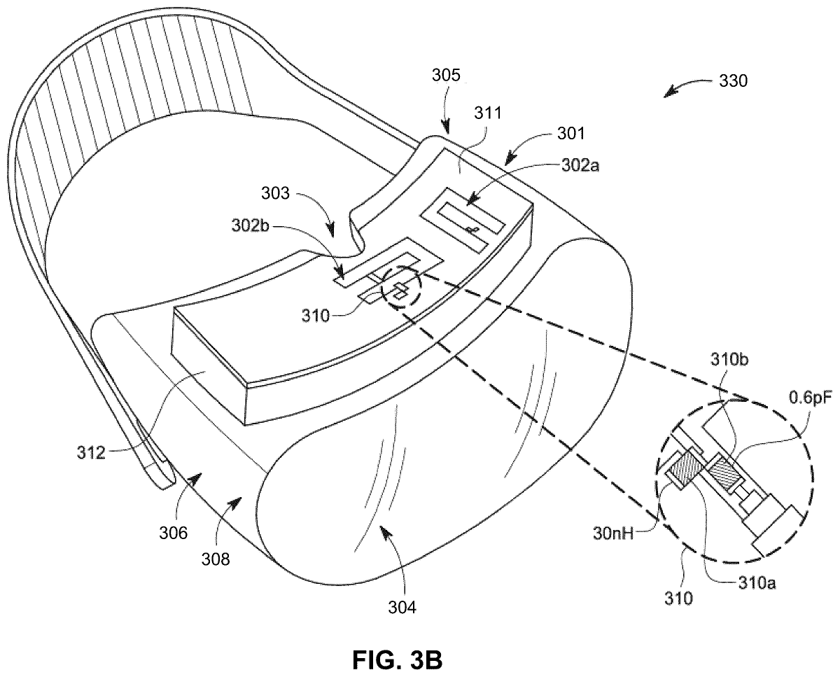

FIG. 3A illustrates an example of a headset device 300 configured to utilize an RF signal obtained via an antenna 302a and an antenna 302b to predict a user's facial/hand pose (in front of the face) configuration, in accordance with some implementations. Antenna 302a and antenna 302b may each comprise a single antenna or multiple antennas. Alternatively, headset device 300 may comprise a single antenna. The example illustrated in FIG. 3 represents a bottom view of headset device 300 with antenna 302a and antenna 302b mounted to a bottom portion 301 of headset device 300. Alternatively, antenna 302a and antenna 302b may each be mounted to or integrated with any portion of headset device 300. Headset device 300 may additionally comprise a vector network analyzer (VNA) 304, a battery 306, a communications module 308, and an impedance matching network 310. Headset device 300 illustrates vector network analyzer (VNA) 304, a battery 306, a communications module 308 as externally mounted components. Alternatively, VNA 304, battery 306, and communications module 308 may be configured as internally mounted components integrated within portions of headset device 300 as illustrated with respect to FIG. 3B, infra.

Headset device 300 may operate as a stand-alone device. Alternatively, headset device 300 may operate in combination with a connected computing device or controller.

Antenna 302a and antenna 302b may each comprise a custom antenna configuration mounted to bottom portion 301 of headset device 300 via a low-profile 3D-printed base (wedge) structure 312 angled slightly towards a face of a user. Alternatively, antenna 302a and antenna 302b may each comprise, inter alia, a cross-polarized antenna system that includes a first vertically-polarized U-shaped slot antenna placed on a center portion 303 of headset device 300 and a second horizontally-polarized antenna placed on a right side portion 305 of headset device 300 (as illustrated in FIG. 3A) such as, for example, a specified distance from an edge of the first vertically-polarized U-shaped slot antenna. The example illustrated with respect to FIG. 3A represents a cross-polarized antenna system that positions antenna 302a and antenna 302b in an orthogonal position with respect to each other. A cross-polarized antenna system utilizing the aforementioned orthogonal positioning may help to reduce environmental noise and may produce a higher signal resolution that may improve capture of multiple different aspects of mouth movements such as, for example, a mouth closed movement, a smile teeth movement, etc. or hand movements such as, for example, finger and joint movements such as, inter alia, fingers moving closer or father from the antenna), etc. Although FIG. 3A illustrates antenna 302a and antenna 302b placed in an orthogonal position with respect to each other, some implementations may include antenna 302a and antenna 302b placed in any type of angular position with respect to each other. Likewise, headset device 300 may comprise an additional antenna(s) placed in a position that is orthogonal to (or placed in any angular configuration) with respect to antenna 302a and/or antenna 302b.

Antenna slots of antenna 302a and antenna 302b may each have specific dimensions that optimize dimensions while providing sufficient sensing capabilities. To reduce an antenna footprint, each of antenna 302a and antenna 302b may be physically folded in half (e.g., lengthwise) thereby maintaining a same electrical length as a straightened slot.

In some implementations, antenna 302a and antenna 302b each may be constructed by utilizing a laser or an alternative cutting device to form antenna base structure 312 comprising a specified shape (for the antenna 302a and antenna 302b) from, for example, an acrylic sheet (e.g., comprising a specified thickness). Subsequently, a copper layer 311 (e.g., copper tape, copper sheet, etc.) may be applied to the antenna base structure 312 such that any voids existing between the copper layer 311 and the antenna base structure 312 are removed during application thereby creating antenna 302a and antenna 302b. A Sub-Miniature Version A (SMA) coaxial cable may be electrically and mechanically connected (e.g., via solder) to antenna 302a and antenna 302b. The SMA coaxial cable may comprise an outer-conductor mesh structure that is electrically and mechanically connected (e.g., via solder) to a ground plane formed by the copper layer. In some implementations, an antenna feed line may be electrically and mechanically connected (e.g., via solder) across antenna slots of antenna 302a and/or antenna 302b.

In an example implementation, an initial self-resonance frequency of antenna 302a and antenna 302b may each be about 2.1 GHz with −23 dB of S11 magnitude. Therefore, to shift an operating frequency of antenna 302a and antenna 302b to be within a close range of a target frequency such as, for example, about 2.5 GHz, impedance matching network 310 may be constructed by connecting a parallel inductor 310a (e.g., about 30 nH) between the antenna feed line and the ground plane. Likewise, a capacitor 310b (e.g., about 0.6 pF) may be serially connected to a port S11 and the antenna feed line.

In some implementations, S11 and S21 parameters may be measured by utilizing VNA 304 mechanically attached to a front portion 300b of headset device 300. VNA 304 may be configured to measure S11 parameters and S21 parameters from a specified frequency range and calculate a return loss magnitude and phase shift. Combined information from the S11 parameters and the S21 parameters may be utilized to estimate fine-grained mouth configurations. VNA 304 may comprise a specialized single or multiple chipset integrated VNA design and may be integrated into a motherboard of headset device 300.

In some implementations, facial and/or hand pose expressions may not require a high frame rate but the facial and/or hand pose expressions may benefit from a higher-resolution sweep to distinguish the facial and/or hand pose expressions from diverse mouth or hand configurations. Therefore, multiple points such as, for example, 61 points may be sampled from a specified frequency range at 5 frames per second (FPS) thereby yielding 61 S11 parameter return loss magnitudes, 61 S11 parameter phase shifts, 61 S21 parameter return loss magnitudes, and 61 S11 parameter phase shifts yielding a total of 244 values that may be passed to a machine learning pipeline for predicting a user facial configuration. Conversely, mouth movements occurring during speech may include rapid movements but may require less resolution to capture a gestalt of mouth movements. In response, 31 points may be sampled from a specified frequency range thereby yielding, for example, 124 values at 8.5 FPS.

In some implementations, calibrations associated with variances between SMA coaxial cables, connectors, fixed dielectrics (e.g., headsets, support wedge, etc.), and additional associated components may be performed such that signal sensitivity is reduced.

Headset device 300 may be configured to operate as a stand-alone device via battery power and wireless functionality. For example, headset device 300 may be configured to operate as a stand-alone device by utilizing communications module 308 to control VNA 304 and by utilizing battery 306 for onboard power.

Headset device 300 may comprise a machine learning pipeline such as machine learning model(s) 210 of FIG. 2, for receiving as input, vectors: S11 return loss magnitudes, S11 phase shifts, S21 magnitudes, S21 phase shifts . . . Snm. Each of the aforementioned vectors may comprise 31 or 61 values in length for a total of 244 values for sensing facial expressions or 144 values for sensing speech movements. In addition to using these raw measurements as machine learning features, we perform additional featurization. For example, for each vector, the following calculations may be executed: a first derivative of a series (61 or 31 features×4), a difference from a previous frame of data (60 or 30×4), a standard deviation (1 feature×4), coefficients from a 3rd order polynomial fit (4 features×4), and subtraction of S11 to S21 vectors (61 or 31 features×2) thereby yielding a total of 870 or 450 features for machine learning.

FIG. 3B illustrates an alternative example of a headset device 330 with respect to headset device 300 of FIG. 3A, in accordance with some implementations. In contrast with headset device 300 of FIG. 3A, headset device 330 of FIG. 3B comprises an alternative internal component configuration with respect to the external component configuration of headset device 300 of FIG. 3A. For example, headset device 330 of FIG. 3B comprises VNA 304, battery 306, and communications module 308 configured as internally mounted components integrated within portions of headset device. VNA 304, communications module 308, and/or battery 306 may comprise a specialized single or multiple chipset integrated design and may be integrated into a specialized printed circuit boards (PCB) and/or a motherboard within headset device 330.

FIG. 4A illustrates an exemplary view 400 in which a user 401 is wearing a headset device 405 enabled to analyze user facial gestures and/or expressions, in accordance with some implementations. Headset device 405 may comprise a same or similar headset device with respect to headset device 300 of FIG. 3A or headset device 330 of FIG. 3B and therefore may include an antenna 402a and an antenna 402b mounted to a bottom portion 417 of headset device 405. Headset device 405 may additionally comprise a vector network analyzer (VNA) 404, a battery 406, a communications module 408, and an impedance matching network 410. VNA 404, battery 406, communications module 408, and impedance matching network 410 may comprise externally mounted components as illustrated in FIG. 3A. Alternatively, VNA 404, battery 406, communications module 408, and impedance matching network 410 may comprise internally mounted components as illustrated in FIG. 3B. Any configuration of internally mounted and externally mounted components may be implemented. Exemplary view 400 illustrates mouth 401a (or facial expression) of user 401 dielectrically and contactlessly interacting with antenna 402 such that changes in a mouth configuration may manifest as changes within self-resonance frequency and performance of the antenna 402. The changes may be measured by headset device 405 and a machine-learning pipeline and/or module may be configured to predict 11 3D key points for cheeks, lips, and tongue of user 401 as illustrated by the phase and magnitude plots 407 for the S11 and S21 parameters. Phase and magnitude plots 407 may be utilized, for example, to configuration a more expressive persona for telepresence uses thereby reducing privacy issues inherent in camera-based systems, while simultaneously supporting (silent) facial expressions that audio-based systems are unable to detect.

FIG. 4B illustrates an exemplary view 419 of a user 420 being presented with facial expressions 424 via a headset device 428 for generating corresponding output personas 432 and associated key points 434, in accordance with some implementations. Generating output personas 432 may utilize a mouth of user 420 dielectrically and contactlessly interacting with an antenna such that changes in a mouth configuration may manifest as changes within self-resonance frequency and performance of the antenna. The facial expressions 424 may include measurements associated with a width of a mouth (e.g., a lip corner to corner) recorded with respect to a neutral configuration (e.g., mean=49.4 mm, SD=3.9) used to scale a 3D head model of user 420 within associated software.

In some implementations headset device 428 may be calibrated by capturing two different datasets to evaluate an overall accuracy of headset device 428. A first dataset includes captured facial expressions, such as facial expressions 424, comprising exaggerated facial movements held for brief periods of time. A second dataset includes mouth movements 425 occurring during speech. The mouth movements 425 occurring during speech may include continuous and rapid small-scale movements.

In some implementations, an accuracy of headset device 428 may be benchmarked by determining a ground truth mouth configuration by utilizing sensors such as cameras to key point a high-resolution video stream of a mouth of user 420. An associated software development kit (SDK) may be configured to output blend-shapes (e.g., 37) utilized to configure a head of user 420 and extract associated key points 434 that may be scaled to match measurements of user 420.

View 419 illustrates facial expressions 424 (e.g., 10) for presentation to user 420 via headset device 428. In response to the presentation, a request to provide facial expressions matching configurations associated with facial expressions 424 is presented to user 420. Resulting facial expressions (e.g., mouth movements 425) provided by user 420 are timestamped for future assessment of discrete configuration classification accuracy.

The request to provide facial expressions may include a first data collection stage that includes requesting all facial expressions (e.g., 10) in a random sequence. The first data collection stage may include five rounds forming one session of data capture (e.g., 5 rounds of 10 facial expressions). During the first data collection stage, synchronized sensor data and ground truth data may be captured continuously (e.g., 5 frames per second (FPS)) to capture a wide variety of intermediate facial configurations between the ten terminal facial expressions (e.g., 10 pairwise combinations) for classification evaluation accuracy.

The request to provide facial expressions may include a second data collection stage that includes collecting mouth movement data resulting from speech. Mouth movement data resulting from speech includes more rapidly changing data than facial expression data. The second data collection stage may include randomly drawn sentences (e.g., 10) being presented, one at a time, to user 420. Each session of the second data collection stage may include 5 rounds of 10 random sentences (e.g., 50 sentence utterances per session). During the second data collection stage, synchronized sensor data and ground truth data may be captured at 8.5 FPS.

In some implementations, multiple training procedures associated with different user calibration and training schemes may be enabled. For example, calibration and training schemes may include, inter alia, across worn headset session accuracy calibration and training, within worn headset session accuracy calibration and training, and across user accuracy calibration and training.

Across headset worn session accuracy calibration and training may include training a model such as machine learning model(s) 210 of FIG. 2 with respect to all but one session of a user data collection using hold out session data for testing. The model training procedure may be implemented for all session data combinations and all results are combined.

Within worn headset session accuracy calibration and training may include training a model such as machine learning model(s) 210 of FIG. 2 with respect to each session of user data collection associated with facial expression and speech movements. In some implementations, each session of data collection may include 5 rounds of data collection thereby allowing for utilization of all data combinations from 4 rounds of data collection for training and 1 round may be used for testing to measure within worn headset session accuracy.

Across user accuracy calibration and training may include training a model such as machine learning model(s) 210 of FIG. 2 with respect to estimating and reporting across-user accuracy by training models with respect to data from all but one user and then use the data from the one user for testing.

In some implementations, facial data associated with a user's facial features may be utilized to uniquely identify the user to, inter alia, load personalized content (e.g., system settings, game progress operations, interpupillary distance, etc.), provide automatic content controls (e.g., for children), etc.

In some implementations, an antenna such as antenna 402 illustrated in FIG. 4A, comprises a low-profile antenna embedded within a surface (e.g., a flat bottom surface such as bottom portion 417) of a headset such that an angle of the antenna may affect performance of the headset. For example, the performance of the headset may be affected because a maximum directivity of a cross-polarized slot antenna system is situated orthogonal to a ground plane of the antenna and therefore, a slight angle of the antenna with respect to a direction toward a mouth of the user may enable a signal to discriminate mouth configurations and reduce resulting noise. Likewise, an antenna array with controllable beam-steering may create an antenna radiation pattern beam tilt such as, for example, 45° with respect to a face of the user.

In some implementations, a direction associated with the antenna may be configured to reduce noise from an environment and associated hand movements. Likewise, the aforementioned tilted radiation beam antenna may be configured to account for a neck orientation such as a nodding-like vertical neck movement that may affect a signal and prediction result. For example, a tilted radiation beam antenna may be configured to precisely aim only at a face rigid body.

FIG. 4C illustrates an exemplary view 438 in which a user 440 is wearing a headset device 441 enabled to analyze user hand (and facial) gestures and/or expressions, in accordance with some implementations. Headset device 441 may comprise a same or similar headset device with respect to headset device 300 of FIG. 3A or headset device 330 of FIG. 3B (and/or headset device 405 of FIG. 4A) and therefore may include an antenna 402a and an antenna 402b mounted to a bottom portion 417 of headset device 441. Headset device 441 may additionally comprise a vector network analyzer (VNA) 404, a battery 406, a communications module 408, and an impedance matching network 410. VNA 404, battery 406, communications module 408, and impedance matching network 410 may comprise externally mounted components as illustrated in FIG. 3A. Alternatively, VNA 404, battery 406, communications module 408, and impedance matching network 410 may comprise internally mounted components as illustrated in FIG. 3B. Any configuration of internally mounted and externally mounted components may be implemented. Exemplary view 438 illustrates a hand pose configuration/expression 449a (e.g., in front of a mouth 443) of user 440 dielectrically and contactlessly interacting with antenna 402a and antenna 402b such that changes in a hand pose configuration may manifest as changes within self-resonance frequency and performance of antenna 402a and antenna 402b. Dielectrically and contactlessly interacting with antenna 402a and antenna 402b may minimize interference generated via face coverings (e.g., a beard, a facemask, etc.) and/or an associated environment by utilizing a directional radiation pattern associated with an angular dependence of a strength of RF waves emitted from antenna 402a and antenna 402b. In some implementations, metallic accessories (e.g., a heavy bracelet or ring on a hand of user 440) may cause interference with RF waves emitted from antenna 402a and antenna 402b to detect changes in a hand pose configuration and therefore, a resulting error message may be presented headset device 441 to indicate that a training process should be executed to retrain headset device 441 while wearing the metallic accessories.

In some implementations, changes in a hand pose configuration may be measured by headset device 405 and a machine-learning pipeline and/or module may be configured to predict 3D key points for a hand, fingers, and joints of user 440 as illustrated by the phase and magnitude plots 451a for the S11 and S21 parameters. Phase and magnitude plots 451a may be utilized, for example, to configure a more expressive persona for telepresence uses thereby reducing privacy issues inherent in camera-based systems, while simultaneously supporting (silent) hand pose expressions that audio-based systems are unable to detect.

FIG. 4D illustrates an alternative exemplary view 457, with respect to exemplary view 438 of FIG. 4C, in which a user 440 is wearing headset device 441 enabled to analyze user hand (and facial) gestures and/or expressions, in accordance with some implementations. In contrast with exemplary view 438 of FIG. 4C, exemplary view 457 of FIG. 4D illustrates a hand pose configuration/expression 449b (e.g., in front of mouth 443) of user 440 representing an emotive cue associated with a silent/quiet gesture. Hand pose configuration/expression 449b comprises changes in hand pose configuration being measured by headset device 405 and a machine-learning pipeline and/or module for predicting 3D key points for hand, fingers, and joints of user 440 as illustrated by the phase and magnitude plots 451b for the S11 and S21 parameters.

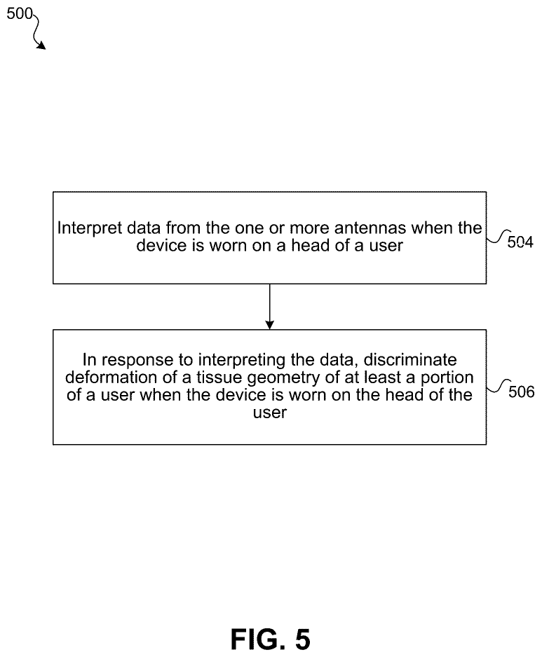

FIG. 5 is a flowchart representation of an exemplary method 500 that utilizes an RF signal obtained via an antenna(s) to predict a user's body portion(s) configuration, in accordance with some implementations. In some implementations, the method 500 is performed by a device, such as a mobile device, desktop, laptop, HMD, or server device. In some implementations, the device has a screen for displaying images and/or a screen for viewing stereoscopic images such as a head-mounted display (HMD such as e.g., device 105 of FIG. 1). In some implementations, the method 500 is performed by processing logic, including hardware, firmware, software, or a combination thereof. In some implementations, the method 500 is performed by a processor executing code stored in a non-transitory computer-readable medium (e.g., a memory). Each of the blocks in the method 500 may be enabled and executed in any order.

The method 500 is performed at a device having a structure, one or more antennas positioned on a portion (for example a bottom portion 301 as illustrated in FIGS. 3A and 3B) of the structure, and a processor coupled to a non-transitory computer-readable storage medium. The one or more antennas may include at least one or combined antenna topologies. The device may be, inter alia, a head-mounted-device (HMD) comprising internal displays, an augmented reality (AR) glasses set comprising transparent eye pieces, a pair of vision corrective glasses, etc.

In some implementations, the one or more antennas may comprise a controllable-beam steering antenna configured to produce a directional radiation pattern. In some implementations, the one or more antennas comprise a slot antenna comprising a slot folded in half. In some implementations, a slot of the one or more antennas may be modified to change a shape or size of the one or more antennas. In some implementations, the one or more antennas may utilize co-polarization or cross-polarization. In some implementations, the one or more antennas may comprise a vertically-polarized U-shaped slot antenna and a horizontally-polarized antenna placed such that the vertically-polarized U-shaped antenna is closer to a central axis of the structure than the horizontally-polarized antenna. In some implementations, the one or more antennas may utilize a 2 port configuration such that each port of the 2 port configuration is configured to inject a signal and measure a reflected signal to its own port and a transmitted signal from the other port. In some implementations, the one or more antennas may provide an antenna radiation pattern beam tilt relative to a face of the user.

At block 504, the method 500 interprets data from the one or more antennas when the device is worn on a head of a user interpreting the data from the one or more antennas may include executing a rule based model or a machine learning model configured to analyze antenna data to track at least one body portion (e.g., a head, a hand, etc.) of a user. For example, user facial features may be tracked for predicting a facial configuration such as, inter alia, 2D or 3D key points with respect to cheeks, lips, jaw, and/or tongue of a user as described in paragraph 0027 with respect to FIG. 2, paragraph 0038 with respect to FIG. 3A, and paragraph 0041 with respect to FIG. 4A. Likewise, user hand pose features may be tracked for predicting and/or discriminating a user's hand pose configurations and associated expressions. The data from the one or more antennas may include, inter alia, return loss magnitudes and phase shifts, additional featurization, etc.

At block 506, the method 500, in response to interpreting the data, discriminates deformation of a tissue geometry of at least a portion (e.g., a body portion) of a user (e.g., a head, a hand, etc.) when the device is worn on the head of the user. In some implementations, the data from the one or more antennas may be associated with one or more impedance characteristics.

In some implementations, interpreting the data and discriminating deformation of at least the portion may include discriminating deformation of a tissue geometry of a head of the user by, inter alia, inputting the data from the one or more antennas to a machine learning or rule based model, outputting 2D or 3D key points corresponding to positions on the surface of the face such as a mouth, tongue, jaw, and/or cheeks of the user, outputting a facial expression classification and/or user identification, accounting for interference from a chest of the user, etc. as described in paragraph 0040 with respect to FIG. 4A.

In some implementations, interpreting the data and discriminating deformation of at least the portion may include discriminating deformation of a hand of the user (e.g., located proximate to a face of the user) by, inter alia, inputting the data from the one or more antennas to a machine learning model, outputting 2D or 3D key points corresponding to positions on the surface of the hand such as a finger, joint, wrist, palm, or a dorsal, etc., outputting a hand expression classification and/or user identification, etc. as described in paragraphs 0057-0059 with respect to FIGS. 4C and 4D.

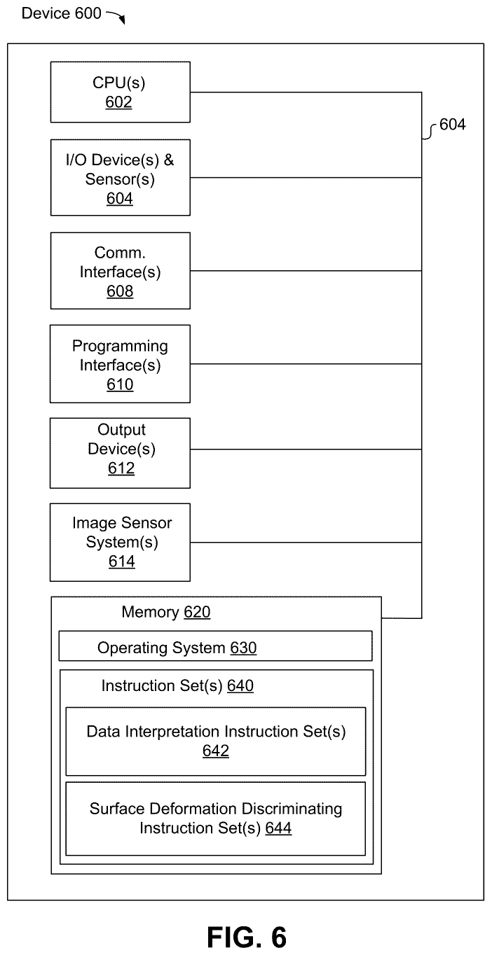

FIG. 6 is a block diagram of an example device 600. Device 600 illustrates an exemplary device configuration for electronic device 105 of FIG. 1. While certain specific features are illustrated, those skilled in the art will appreciate from the present disclosure that various other features have not been illustrated for the sake of brevity, and so as not to obscure more pertinent aspects of the implementations disclosed herein. To that end, as a non-limiting example, in some implementations the device 600 includes one or more processing units 602 (e.g., microprocessors, ASICs, FPGAs, GPUs, CPUs, processing cores, and/or the like), one or more input/output (I/O) devices and sensors 604, one or more communication interfaces 608 (e.g., USB, FIREWIRE, THUNDERBOLT, IEEE 802.3x, IEEE 802.11x, IEEE 802.14x, GSM, CDMA, TDMA, GPS, IR, BLUETOOTH, ZIGBEE, SPI, I2C, and/or the like type interface), one or more programming (e.g., I/O) interfaces 610, output devices (e.g., one or more displays) 612, one or more interior and/or exterior facing image sensor systems 614, a memory 620, and one or more communication buses 604 for interconnecting these and various other components.

In some implementations, the one or more communication buses 604 include circuitry that interconnects and controls communications between system components. In some implementations, the one or more I/O devices and sensors 606 include at least one of an inertial measurement unit (IMU), an accelerometer, a magnetometer, a gyroscope, a thermometer, one or more physiological sensors (e.g., blood pressure monitor, heart rate monitor, blood oxygen sensor, blood glucose sensor, etc.), one or more microphones, one or more speakers, a haptics engine, one or more depth sensors (e.g., a structured light, a time-of-flight, or the like), one or more cameras (e.g., inward facing cameras and outward facing cameras of an HMD), one or more infrared sensors, one or more heat map sensors, and/or the like.

In some implementations, the one or more displays 612 are configured to present a view of a physical environment, a graphical environment, an extended reality environment, etc. to the user. In some implementations, the one or more displays 612 are configured to present content (determined based on a determined user/object location of the user within the physical environment) to the user. In some implementations, the one or more displays 612 correspond to holographic, digital light processing (DLP), liquid-crystal display (LCD), liquid-crystal on silicon (LCoS), organic light-emitting field-effect transitory (OLET), organic light-emitting diode (OLED), surface-conduction electron-emitter display (SED), field-emission display (FED), quantum-dot light-emitting diode (QD-LED), micro-electromechanical system (MEMS), and/or the like display types. In some implementations, the one or more displays 612 correspond to diffractive, reflective, polarized, holographic, etc. waveguide displays. In one example, the device 600 includes a single display. In another example, the device 600 includes a display for each eye of the user.

In some implementations, the one or more image sensor systems 614 are configured to obtain image data that corresponds to at least a portion of the physical environment 100. For example, the one or more image sensor systems 614 include one or more RGB cameras (e.g., with a complimentary metal-oxide-semiconductor (CMOS) image sensor or a charge-coupled device (CCD) image sensor), monochrome cameras, IR cameras, depth cameras, event-based cameras, and/or the like. In various implementations, the one or more image sensor systems 614 further include illumination sources that emit light, such as a flash. In various implementations, the one or more image sensor systems 614 further include an on-camera image signal processor (ISP) configured to execute a plurality of processing operations on the image data.

In some implementations, sensor data may be obtained by device(s) (e.g., devices 105 of FIG. 1) during a scan of a room of a physical environment. The sensor data may include a 3D point cloud and a sequence of 2D images corresponding to captured views of the room during the scan of the room. In some implementations, the sensor data includes image data (e.g., from an RGB camera), depth data (e.g., a depth image from a depth camera), ambient light sensor data (e.g., from an ambient light sensor), and/or motion data from one or more motion sensors (e.g., accelerometers, gyroscopes, IMU, etc.). In some implementations, the sensor data includes visual inertial odometry (VIO) data determined based on image data. The 3D point cloud may provide semantic information about one or more elements of the room. The 3D point cloud may provide information about the positions and appearance of surface portions within the physical environment. In some implementations, the 3D point cloud is obtained over time, e.g., during a scan of the room, and the 3D point cloud may be updated, and updated versions of the 3D point cloud obtained over time. For example, a 3D representation may be obtained (and analyzed/processed) as it is updated/adjusted over time (e.g., as the user scans a room).

In some implementations, sensor data may be positioning information, some implementations include a VIO to determine equivalent odometry information using sequential camera images (e.g., light intensity image data) and motion data (e.g., acquired from the IMU/motion sensor) to estimate the distance traveled. Alternatively, some implementations of the present disclosure may include a simultaneous localization and mapping (SLAM) system (e.g., position sensors). The SLAM system may include a multidimensional (e.g., 3D) laser scanning and range-measuring system that is GPS independent and that provides real-time simultaneous location and mapping. The SLAM system may generate and manage data for a very accurate point cloud that results from reflections of laser scanning from objects in an environment. Movements of any of the points in the point cloud are accurately tracked over time, so that the SLAM system can maintain precise understanding of its location and orientation as it travels through an environment, using the points in the point cloud as reference points for the location.

In some implementations, the device 600 includes an eye tracking system for detecting eye position and eye movements (e.g., eye gaze detection). For example, an eye tracking system may include one or more infrared (IR) light-emitting diodes (LEDs), an eye tracking camera (e.g., near-IR (NIR) camera), and an illumination source (e.g., an NIR light source) that emits light (e.g., NIR light) towards the eyes of the user. Moreover, the illumination source of the device 600 may emit NIR light to illuminate the eyes of the user and the NIR camera may capture images of the eyes of the user. In some implementations, images captured by the eye tracking system may be analyzed to detect position and movements of the eyes of the user, or to detect other information about the eyes such as pupil dilation or pupil diameter. Moreover, the point of gaze estimated from the eye tracking images may enable gaze-based interaction with content shown on the near-eye display of the device 600.

The memory 620 includes high-speed random-access memory, such as DRAM, SRAM, DDR RAM, or other random-access solid-state memory devices. In some implementations, the memory 620 includes non-volatile memory, such as one or more magnetic disk storage devices, optical disk storage devices, flash memory devices, or other non-volatile solid-state storage devices. The memory 620 optionally includes one or more storage devices remotely located from the one or more processing units 602. The memory 620 includes a non-transitory computer readable storage medium.

In some implementations, the memory 620 or the non-transitory computer readable storage medium of the memory 620 stores an optional operating system 630 and one or more instruction set(s) 640. The operating system 630 includes procedures for handling various basic system services and for performing hardware dependent tasks. In some implementations, the instruction set(s) 640 include executable software defined by binary information stored in the form of electrical charge. In some implementations, the instruction set(s) 640 are software that is executable by the one or more processing units 602 to carry out one or more of the techniques described herein.

The instruction set(s) 640 includes a data interpretation instruction set 642 and a surface deformation tracking instruction set 644. The instruction set(s) 640 may be embodied as a single software executable or multiple software executables.

The data interpretation instruction set 642 is configured with instructions executable by a processor to interpret data from one or more antennas.

The surface deformation discriminating instruction set 644 is configured with instructions executable by a processor to track deformation of a tissue geometry of a head or a hand (located proximate to a mouth) of user when a device is worn on the head of the user.

Although the instruction set(s) 640 are shown as residing on a single device, it should be understood that in other implementations, any combination of the elements may be located in separate computing devices. Moreover, FIG. 6 is intended more as functional description of the various features which are present in a particular implementation as opposed to a structural schematic of the implementations described herein. As recognized by those of ordinary skill in the art, items shown separately could be combined and some items could be separated. The actual number of instructions sets and how features are allocated among them may vary from one implementation to another and may depend in part on the particular combination of hardware, software, and/or firmware chosen for a particular implementation.

Those of ordinary skill in the art will appreciate that well-known systems, methods, components, devices, and circuits have not been described in exhaustive detail so as not to obscure more pertinent aspects of the example implementations described herein. Moreover, other effective aspects and/or variants do not include all of the specific details described herein. Thus, several details are described in order to provide a thorough understanding of the example aspects as shown in the drawings. Moreover, the drawings merely show some example embodiments of the present disclosure and are therefore not to be considered limiting.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable sub combination. Moreover, although features may be described above as acting in certain combinations and even initially claimed as such, one or more features from a claimed combination can in some cases be excised from the combination, and the claimed combination may be directed to a sub combination or variation of a sub combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, e.g., one or more modules of computer program instructions, encoded on computer storage medium for execution by, or to control the operation of, data processing apparatus. Alternatively, or additionally, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. Unless specifically stated otherwise, it is appreciated that throughout this specification discussions utilizing the terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or display devices of the computing platform.

The system or systems discussed herein are not limited to any particular hardware architecture or configuration. A computing device can include any suitable arrangement of components that provides a result conditioned on one or more inputs. Suitable computing devices include multipurpose microprocessor-based computer systems accessing stored software that programs or configures the computing system from a general purpose computing apparatus to a specialized computing apparatus implementing one or more implementations of the present subject matter. Any suitable programming, scripting, or other type of language or combinations of languages may be used to implement the teachings contained herein in software to be used in programming or configuring a computing device.

Implementations of the methods disclosed herein may be performed in the operation of such computing devices. The order of the blocks presented in the examples above can be varied for example, blocks can be re-ordered, combined, and/or broken into sub-blocks. Certain blocks or processes can be performed in parallel. The operations described in this specification can be implemented as operations performed by a data processing apparatus on data stored on one or more computer-readable storage devices or received from other sources.

The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or value beyond those recited. Headings, lists, and numbering included herein are for ease of explanation only and are not meant to be limiting.

It will also be understood that, although the terms “first,” “second,” etc. may be used herein to describe various elements, these elements should not be limited by these terms. These terms are only used to distinguish one element from another. For example, a first node could be termed a second node, and, similarly, a second node could be termed a first node, which changing the meaning of the description, so long as all occurrences of the “first node” are renamed consistently and all occurrences of the “second node” are renamed consistently. The first node and the second node are both nodes, but they are not the same node.

The terminology used herein is for the purpose of describing particular implementations only and is not intended to be limiting of the claims. As used in the description of the implementations and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” may be construed to mean “upon determining” or “in response to determining” or “in accordance with a determination” or “upon detecting” or “in response to detecting” that the stated condition precedent is true, depending on the context.

What is claimed is:

1. A device comprising:
a structure;
one or more antennas positioned on a portion of the structure, wherein the one or more antennas comprises at least one or combined antenna topologies; and
a processor configured to interpret data from the one or more antennas to discriminate deformation of a tissue geometry of at least a portion of a user when the device is worn on the head of the user.

2. The device of claim 1, wherein the data from the one or more antennas is associated with one or more impedance characteristics.

3. The device of claim 1, wherein the one or more antennas utilize a 2 port configuration, wherein each port of the 2 port configuration is configured to inject a signal and measure a reflected signal to its own port and a transmitted signal from the other port.

4. The device of claim 1, wherein the at least a portion comprises a head of the user.

5. The device of claim 4, wherein the one or more antennas provide an antenna radiation pattern beam tilt relative to a face of the user.

6. The device of claim 5, wherein the data from the one or more antennas comprises return loss magnitudes and phase shifts.

7. The device of claim 4, wherein the data from the one or more antennas comprises additional featurization.

8. The device of claim 4, wherein interpreting data from the one or more antennas to discriminate deformation of the tissue geometry of the at least a portion of the user comprises inputting the data from the one or more antennas to a machine learning model.

9. The device of claim 4, wherein said interpreting data from the one or more antennas to discriminate deformation of the tissue geometry of the at least a portion of the user comprises outputting 2D or 3D key points corresponding to positions on a surface of a face of the user.

10. The device of claim 9, wherein the 2D or 3D key points are on a mouth, tongue, jaw, or cheeks of the user.

11. The device of claim 4, wherein interpreting data from the one or more antennas to discriminate deformation of the tissue geometry of the at least a portion of the user comprises outputting a facial expression classification.

12. The device of claim 4, wherein interpreting data from the one or more antennas to discriminate deformation of the tissue geometry of the at least a portion of the user comprises outputting a user identification.

13. The device of claim 4, wherein interpreting data from the one or more antennas to discriminate deformation of the tissue geometry of the at least a portion of the user comprises accounting for interference from a chest of the user.

14. The device of claim 1, wherein the at least a portion comprises at least one hand of the user located proximate to a face of the user.

15. The device of claim 14, wherein interpreting data from the one or more antennas to discriminate geometry of the at least a portion of the user comprises outputting 2D or 3D key points corresponding to positions on a surface of the at least one hand.

16. The device of claim 15, wherein the 2D or 3D key points are on a finger, joint, wrist, palm, or dorsal of the at least one hand of the user.

17. The device of claim 14, wherein interpreting data from the one or more antennas to discriminate deformation of the tissue geometry of the at least a portion of the user comprises outputting a hand expression classification.

18. The device of claim 14, wherein interpreting data from the one or more antennas to discriminate deformation of the tissue geometry of the at least a portion of the user comprises outputting a user identification.

19. A method comprising:
at a device having a structure, one or more antennas, comprising at least one or combined antennas topologies, positioned on a portion of the structure, and a processor coupled to a non-transitory computer-readable storage medium:
interpreting data from the one or more antennas when the device is worn on a head of a user; and
in response to said interpreting the data, discriminating deformation of a tissue geometry of at least a portion of a user when the device is worn on the head of the user.

20. A non-transitory computer-readable storage medium, storing program instructions executable by one or more processors to perform operations comprising: at a device having a structure, one or more antennas, comprising at least one or combined antennas topologies, positioned on a portion of the structure, and a processor coupled to a non-transitory computer-readable storage medium:
interpreting data from the one or more antennas when the device is worn on a head of a user; and
in response to said interpreting the data, discriminating deformation of a tissue geometry of at least a portion of a user when the device is worn on the head of the user.
